(19) United States
(12) Patent Application Publication
(10) Pub. No.: US 2005/0223440 A1
(43) Pub. Date: Oct. 6, 2005

(54) Tissue culture process for producing cotton plants

(75) Inventors: Rakesh Tuli, New Delhi (IN); Mithilesh Kumar, New Delhi (IN)

Correspondence Address: Ladas & Parry, 26 West 61 Street, New York, NY, 10023, US

(73) Assignee: Council of Scientific and Industrial Research

(21) Appl. No.: 10/815,108

(22) Filed: Mar. 31, 2004

(51) Int. Cl. A01H 5/00; C12N 15/82

(52) U.S. Cl. 800/314; 435/468

(57) ABSTRACT

A method for plant regeneration in cotton via developmentally synchronized somatic embryogenesis is disclosed. The invention is simple, fast, reproducible and convenient for applications in plant genetic engineering and results in the achievement of synchronised somatic embryogenesis by a step of inositol starvation.

### FIELD OF THE INVENTION

The present invention relates to a tissue culture process for producing a large number of viable cotton plants in vitro from a specified tissue of the plant. The invention provides a method for synchronized somatic embryogenesis and opens up new possibilities for obtaining agronomically improved uniform population of cotton plants by modern methods of agrobiotechnology and genetic engineering. The protocol provides an important step in the success of cotton improvement programme utilizing tissue culture technology.

### BACKGROUND OF THE INVENTION

Cotton is a globally important crop, grown primarily for fiber. Seeds provide an important source of food for livestock. Cotton has influenced the economic development of many nations throughout the world. Therefore, cotton improvement programmes by modern methods of agrobiotechnology are of interest worldwide. This has increased the importance of developing tissue culture methods to facilitate the application of modern techniques of genetic engineering to cotton plant. In spite of the much-talked economic value of cotton, the improvement of cotton through genetic engineering has taken place at a relatively slow rate because of the absence of reproducible, less time consuming and efficient methods to regenerate organized tissues and plants of cotton at high frequency.

Plant regeneration by tissue culture techniques is well established. Although the totipotency of a plant cell is a well known phenomenon, each plant or plant part requires specialized studies to invent the conditions that allow such regeneration at high efficiency and frequency. There seems to be a consensus that success in inducing differentiation depends upon the type of explant, physiological condition of the explant and physical and chemical milieu of the explant during culture. Thus, the science of tissue culture has been directed to optimize the physiological condition of source plant, the type of explant, the culture conditions and the plant growth regulators or other media addenda used to initiate the tissue response.

A wide variety of plant species has been successfully regenerated in in vitro organogenesis or via somatic embryogenesis. The mode of regeneration selected is often based on the relative case, efficiency and applicability of the method for genetic transformation of a plant species. A non meristem based method i.e. somatic embryogenesis is always a preferred mode, as it eliminates the possibility of getting false positive or chimaeric transformants.

The regeneration via somatic embryogenesis from an explant may involve several growth stages. Most often, an explant from a mature plant part or organ or from germinated seedling is given a chemically defined nutrient medium under sterile conditions. Upon incubation, the excised plant part under artificially controlled conditions of light, temperature and photoperiod gives rise to a dedifferentiated mass of cells, referred to as a callus.

As a result of culturing the callus under proper set of physical and chemical milieu i.e. nutrients, light, temperature, photoperiod and by adding appropriate combination and concentration of plant growth regulators, or by suddenly removing these, the calli of some plant species have been reported to generate embryogenic callus which, in turn, is subjected to form somatic embryos, in a process called somatic embryogenesis.

Somatic embryos are embryos developed out of somatic cells. Each somatic embryo is an organized mass of tissue capable of developing into a complete plant. Somatic embryos are very similar to zygotic embryos developed in seed, except that they develop without involvement of reduction cell division (meiosis) and they are often bigger in size.

Changes in the concentration of one or more medium constituents may lead to changes in the in-vitro development and differentiation of plant tissue. Signaling pathways mediated by phosphoinositols have been reported to influence development and embryogenesis in plants. Starving the tissues at a particular stage for inositol for a definite period of time may produce synchronization of development of tissues without deteriorating their viability. However, involvement of inositol in achieving synchronisation of plant or embryo development in vitro has not been reported earlier and is the most critical aspect of this invention.

As of now, several reports on cotton somatic embryogenesis techniques have been published/patented. The frequency of somatic embryogenesis among the explants over a defined treatment is typically low. Moreover, the number of embryos obtained per explant is not high. Long time period required for in vitro embryogenesis and genotype dependency of the process further add to the problem of somatic embryogenesis in cotton.

It would be advantageous if a protocol can be developed even for a model cultivar of cotton to provide a set of formulation and conditions by which the frequency of somatic embryogenesis can be improved and if such a process needs less time. The synchronized development can be beneficial in harvesting a larger number of regenerated plants. Synchronized development gives not only large number of plants but also uniform population with all plants in same phase of growth. Simplifying the steps and formulations can further enhance the applicability of the protocol. Using liquid medium can further facilitate efficient selection during genetic transformation (transgenic plant development) since the selection pressure (for example, resistance to antibiotics) can penetrate the cells more uniformly in liquid culture. All these aspects have been achieved in the present invention. Synchronized development of embryos in cotton has not been mentioned in prior art and is an important aspect of this invention.

### DESCRIPTION OF THE PRIOR ART

There are several published reports dealing with tissue culture conditions, which lead to embryogenesis and plant regeneration in cotton. Davidonis and Hamilton, in Plant Sci. Letter (1983) 32:89-93 and in U.S. Pat. No. 4,672,035 (1987) report somatic embryogenesis in 2 years old calli of G. hirsutum. Shoemaker et al., 1986 characterized somatic embryogenesis and plant regeneration in 17 cultivars of cotton (Plant Cell Rep. 3:178-181). Trolinder and Goodin (1987) and Finer (1988) reported somatic embryogenesis in suspension culture of cotton (Plant Cell Rep 6:231-234; Plant Cell Rep. 7:399-402). These protocols took several months to develop regenerated plants. The methods are highly dependent on genotypes (Trolinder and Xhixian, 1989 Plant Cell Rep. 8:133-136) and therefore, applicable to only a few cultivars of cotton. Due to lengthy culture time, plants developed via these protocols were often reported to be sterile or having cytogenetic abnormalities (Trolinder and Goodin, 1987 Plant Cell Rep. 6:231-234). Rangan (1993) in U.S. Pat. No. 5,244,802 and Rangan and Rajasekaran (1997) in U.S. Pat. No. 5,695,999 reported embryogenesis from seedling explants in several varieties of cotton. Gawel and Robacker (1990) in Plant Cell Tiss. Organ Cult. 23:201-204 compared somatic embryogenesis in cotton on semi-solid versus liquid proliferation media. Kumar et al., 1998 reported somatic embryogenesis in F1 hybrids of Coker 310 with Indian varieties of cotton utilizing modified Trolinder and Goodin protocol. Zhang et al., 2000 in Plant Cell Tiss. Organ Cult. 60:89-94 described somatic embryogenesis from abnormal somatic embryo derived explants.

Several of these protocols or modifications thereof have been used in Agrobacterium mediated transformation of cotton (Umbeck et al., 1987 Bio/Technology 5: 263-266; Firoozabady et al., 1987 Plant Mol. Biol. 10: 105-116) or particle bombardment mediated transformation of cotton (Finer and McMullen, 1990 Plant Cell Rep. 8:586-589). Certain bacterial genes, like those encoding herbicide resistance (Bayley et al., 1992 Theo. Appl. Genet. 83: 645-649) and Bacillus thuringiensis endotoxin genes (Perlak et al., 1990 Bio/Technology 8: 939-943) have been successfully expressed in transgenic plants. Strickland (1998) in U.S. Pat. No. 5,846,797 reported regeneration from transformed explants on growth regulator free medium. Once an efficient method for regeneration, specially somatic embryogenesis becomes available, it can conveniently be used for transformation or genetic engineering of cotton.

A comparative chart between various prior art is presented below as Table 1:

TABLE 1
Review of prior art on regeneration in cotton with an aim to induce somatic emryogenesis.

| | | | | Development | |
|---|---|---|---|---|---|
| | Report | Explant | Phytohormones | synchnrony | Remarks |
| 1. | Davidonis GH and Hamilton RH | Cotyledon | NAA, Kin. | — | 2 years old calli of G. hirsutum L. cv. Coker 310 |
| | (1983) Plant regeneration from callus | | | | grown on LS medium containing 30 gm/L |
| | of G. hirsutum L. Plant Sci. Lett. 32: | | | | glucose in absence of NAA and kin were used |
| | 89-93 | | | | 30% cultures gave rise to somatic embryos. |
| 2. | Davidonis GH, Mumma RO, Hamilton | ″ | ″ | — | 2 years old calli of G. hirsutum L. cv. Coker 310 |
| | RH (1987) Controlled regeneration of | | | | grown on LS medium containing 30 gm/L |
| | cotton plants from tissue culture U.S. | | | | glucose in absence of NAA and kin were used |
| | Pat. No. 4672035 | | | | 30% cultures gave rise to somatic embryos. |
| 3. | Shoemaker RC, Couche LS and | Hypocotyl | NAA, Kin. | — | 17 Cultivars of cotton G. hirsutum L. were |
| | Galbraith D.W. (1986) | | | | evaluated for somatic embryogenesis. After a |
| | Characterization of somatic | | | | series of transfer of calli through medium |
| | embryogenesis and plant regeneration | | | | containing MS salto, NAA & Kin. for several |
| | in cotton Gossypium hirsutum L. Plant | | | | weeks, calli were observed for the presence of |
| | Cell Rep. 3: 178-181. | | | | somatic embryos. Cultivars Coker 201 and Coker |
| | | | | | 315 were identified as embryogenic. The |
| | | | | | embryos were isolated and developed into plants. |
| 4. | Trolinder NL and Goodin JR (1987) | Hypocotyl | 2,4D, Kin. | — | Globular embryos were observed in 6 weeks old |
| | Somatic embryogenesis and plant | | | | callus culture. At this stage calli were |
| | regeneration in cotton Gossypium | | | | subcultured to liquid suspension in growth |
| | hirsutum L. Plant Cell Rep. 6: 231-234 | | | | regulator free medium. After 3-4 weeks |
| | | | | | suspensions were sieved to collect globular and |
| | | | | | heart stage embryos. Collected embryos were |
| | | | | | developed on solidified medium to maturity. |
| | | | | | Mature embryos were germinated into plants. |
| | | | | | Most of the plants developed by this method |
| | | | | | were sterile. (only 15% plants were fertile). |
| 5. | Trolinder NL and Xhixian C (1989) | Hypocotyl | 2, 4D, Kin. | — | 38 Cultivars, strains and races of Gossypium |
| | Genotype specificity of somatic | | | | were screened for somatic embryogenesis with |
| | embryogenesis response in cotton. | | | | the method developed for Coker 312. Screening |
| | Plant Cell Rep. 8: 133-136 | | | | indicated that genotype variation for |
| | | | | | embryogenesis existed. Only a few genotype are |
| | | | | | amenable to the model developed for Coker 312. |
| 6. | Finer JJ (1988) Plant regeneration | Cotyledon | NAA, Pictoram | | Maintainable embryogenic suspension cultures |
| | from somatic embryogenic suspension | | 2, 4D, Kin, | | were developed. Embryos were developed by |
| | cultures of cotton (Gossypium | | | | transferring embryogenic tissue to auxin free |
| | hirsutum L). Plant Cell Rep. 7: 399-402 | | | | medium. Plants derived were fertile. |
| 7. | Finer JJ (1990) An efficient method for | ″ | NAA, Pictoram | — | Maintainable embryogenic suspension cultures |
| | regenerating cotton from cultured | | 2, 4D, Kin, | | were developed. Embryos were developed by |
| | cells. Patent no. ZA 48808599 | | | | transferring embryogenic tissue to auxin free |
| | | | | | medium. Plants derived were fertile. |
| 8. | Rangan TS (1993) Regeneration of | Hypocotyl | NAA, Kin. | — | Callus was initiated on MS medium containing |
| | cotton U.S. Pat. No. 5244802 | Cotyledon | | | NAA and Kin., subeultured every 3^{rd} week for |
| | | Immature | | | growth. Somatic embryos were formed four to |
| | | embryo | | | six month after placing tissue on callus initiation |
| | | | | | medium. Varieties identified embryogenic in |
| | | | | | vitro are SJ2, SJ14, SJ5, SJ2C, GC510, B1644, |
| | | | | | B2710, Siokra and FC 2017. |
| 9. | Rangan TS and Rajasekaran K (1997) | Hypocotyl | NAA, Kin. | — | Calli formed over explants on semi-solid callus |
| | Regeneration of cotton plant in | Cotyledon | | | induction medium containing glucose NAA & |
| | suspension culture. U.S. Pat. No. | & immature | | | kin. were proliferated in liquid callus growth |
| | 5,695,999 | embryo | | | medium containing sucrose and NAA. Somatic |
| | | | | | embryos formed on this medium were |
| | | | | | germinated on Beasley & Ting's medium. |
| 10. | Gawel NJ and Robacker CD (1990) | Petiole | 2, 4D Kin. | — | A comparative study was made for somatic |
| | Somatic embryogenesis in two | from | | | embmyogenesis in liq. versus solid media and it |
| | Gossypium hirsutum genotypes on | mature | | | was found that culture on liquid media favours |
| | semi-solid vs liquid proliferation | plants | | | the somatic embryogenesis in both the genotypes |
| | media. Plant Cell Tissue Organ Culture | | | | named Coker 312 and T25. |
| | 23: 201-204. | | | | |
| 11. | Kumar S, Sharma P and Pental D. A | Hypocotyl | 2, 4D, Kin. | — | Use of Trolinder and Goodin (Protocol) on solid |
| | genetic approach to in vitro | | | | media with modification (inclusion of A/C for |
| | regeneration of non-regenerating | | | | maturation of embryos) gave somatic |
| | cotton (Gossypium hirsutum L.) | | | | embryogenesis in Coker 310 and Fl hybrids of |
| | cultivars. | | | | Coker 310 with Indian varieties namely MCU5. |
| | | | | | MCU7, Khandwa-2. Bikaneri Nurma and F486. |
| 12. | Zhang B-H, Liu F and Yao C-B (2000) | Leaf | IAA, 2, 4D, Zt. | — | Embryogenic callus and somatic embryos were |
| | Plant regeneration via somatic | pieces | | | obtained directly from the explants of abnormal |
| | embryogenesis in cotton. Plant Cell | and | | | cotton seedlings on MS medium supplemented |
| | Tissue and Organ Culture 60: 89-94. | stem | | | with Zt and A/C in varieties Coker 201 and CRI |
| | | sections | | | 12. |
| 13. | Umback P, Johnson G, Barton K & | Hypocotyl | 2, 4D, Kin, | — | Method of genetic transformation of cotton was |
| | Swain W (1987). Genetically | | | | disclosed. Immature tissue of cotton was |
| | transformed cotton (Gossypium | | | | transformed in vitro via Agrobacterium. The |
| | hirsutum) plant. Bio/technology 5: | | | | resulting cotton tissues were screened for |
| | 263-266. | | | | transformation by selection on antibiotic. |
| | | | | | Transformed cultures were then induced to give |
| | | | | | somatic embryos. The somatic embryos were |
| | | | | | developed into mature plants. Coker 310, 312 |
| | | | | | and 5110 were transformed by this method. |
| 14. | Firoozabady E., DeBoer LD, Merlo | Cotyledon | 2ip, NAA | — | Cotyledon explants from 12 d old seedlings were |
| | JD, Halk LE, Rashka KE and Murrery | | | | transformed and plants were regenerated for |
| | EE (1987). Transformation of cotton | | | | regeneration the explant treated with |
| | Gossypium hirsutum L. by | | | | Agrobacterium were transferred to medium 28 |
| | Agrobacterium tumefaciens and | | | | containing antibiotics so that only transformed |
| | regeneration of transgenic plants. Plant | | | | cells give rise to callus. Callus when subcultured |
| | Mol. Biol. 10: 105-116. | | | | to hormone free medium gave rise to transgenic |
| | | | | | plants. Using this method G. hirsutum Coker 201 |
| | | | | | was transformed. |
| 15. | Perlak FJ, Deaton RW, Armstrong TA, | Hypocotyl | 2, 4D | — | Truncated forms of insect control protein genes |
| | Fuchs RL, Stins SR. Greenplate and | | | | of Bacillus thuringiensis var. Kurstaki HD-1 |
| | Fischoff DAs (1990) Insect resistant | | | | [Cry1A(b) and HD73, Cry1A(c)] were |
| | cotton plants Bio/Technology 8: 939-943 | | | | transformed into cotton hypocotyl segments via |
| | | | | | Agrobacterium tumefaciens and somatic embryos |
| | | | | | were obtained from transformed cells and finally |
| | | | | | insect resistant cotton plants of G. hirsutum cv. |
| | | | | | Coker 312 were obtained. |
| 16. | Bayley C, Trolinder NL, Ray C, | Hypocotyl | 2, 4D, Kin. | — | 2, 4D monoxygenase gene tfd A from |
| | Morgan M, Quisenberry JE and OW | | | | Alicaligenus europhus ws isolate, modified and |
| | DW. (1992) Engineering 2. 4D | | | | expressed in tobacco and cotton plants. |
| | resistance into cotton. Theo. App. | | | | Transformation was done by A. tumefaciens and |
| | Genet. 83: 645-649. | | | | 2, 4D resistant plants of Coker 312 line were |
| | | | | | obtained. |
| 17. | Rangan TS, Raja-sekaran K, Hudspeth | Hypocotyl | NAA, Kin. | — | Regeneration and transformation of cotton (G. |
| | and Yenofsky (1989) Regeneration and | cotyledon | | | hirsutum var. Accul SJ2, SJ4. SJ5, SJ2-1, |
| | transformation of cotton. Patent no. | & immature | | | GC510, B1644, B2724, B1810, Picker variety of |
| | EP344302. | embryo | | | siokra and stipper var. FC 2017); transformation |
| | | | | | was done by A. Tumefaciens, somatic embryos |
| | | | | | obtained from callus were germinated on Beasley |
| | | | | | and Ting's medium. |
| 18. | Finer JJ and Mc Mullen (1990) | Cotyledon | NAA, Kin, 2, | — | Embryogenic suspension cultures of cotton were |
| | Transformation of cotton Gossypium | | 4D & Picloram | | subjected to particle bombardment where high |
| | hirsutum L. via particle bombardment. | | | | density particles carrying plasmid DNA were |
| | Plant Cell Rep. 8: 586-589. | | | | accelerated towards the embryogenic plant cell. |
| | | | | | These cells were then subjected to developmental |
| | | | | | process of somatic embryogenesis in presence of |
| | | | | | antibiotic (Hygromycin) Coker 3 10 was |
| | | | | | transformed by this method. |
| 19. | Strickland SG (1998) | Hypocotyl | — | — | Explants were transformed with Agrobacterium |
| | | tissue | | | and transformed explants cultured on growth |
| | | | | | regulator free medium directly gave rise to |
| | | | | | embryogenic callus which is cultured further to |
| | | | | | produce somatic embryos. Varieties used were |
| | | | | | Coker 320, 9358 & 84-828. |

The report 4 of Table 1 claims the development of plants that were sterile while the present process described in this application gives healthy plants, which are fertile. The present method gives a high frequency of somatic embryogenesis over randomly selected pool of seedling explants collected from field grown plants. This is an important improvement over the earlier reports where explants were taken from samples already selected for somatic embryogenesis (reports 11 and 12 of table 1). Furthermore, as of now, there is no published report describing synchronization in somatic embryogenesis of cotton. There is no report at all for any other plant, whatsoever, describing inositol starvation as a tool to induce such synchrony in the development of somatic embryos. In case of cotton, synchronized development of somatic embryos has not been possible in prior art and is a very important achievement of this invention.

The success of the present invention depends upon the concentration and combination of plant growth regulators utilized to induce calli over the explants and the manner in which these calli were cultured by the method of the present invention involving a short term inositol deprivation phase. Once the calli have been induced, the process does not need any exogenous plant growth regulator or other medium additives (For example, extra KNO₃ in reports 4 and 6 and activated charcoal in reports 11 and 12) in any subsequent steps. The somatic embryos germinated and rooted in a simplified liquid germination medium on a non-gelling agent based in expensive and simple support (example, vermiculite). In this respect, the present invention describes a simple and less expensive protocol, suitable for commercialisation.

The present invention for the first time describes that inositol influences the development and differentiation of in-vitro grown plant cells. The method details the use of inositol deprivation of cultures at and for a particular time for the synchronized development of somatic embryos. When selected embryogenic clumps of cells were subjected to a 8-12 days period of inositol starvation and cultures were thereafter returned to basal medium containing inositol, nearly all embryos were found in globular stage. After 10 days of further growth, maximum embryos (92%) were in heart stage. And in subsequent subculturing approximately 82% of embryos were in torpedo stage. When embryogenic clumps were subjected to 2 cycles of 8-12 days of inositol starvation, the said synchronization of development was not observed after globular stage. Moreover, this short-term inositol starvation not only synchronized the development of embryos, the number of embryos finally recovered surprisingly increased to a 4-5 fold higher value.

The earlier patents in this area on cotton are U.S. Pat. No. 4,672,035; U.S. Pat. No. 5,244,802; U.S. Pat. No. 5,695,999; EP 344302 and U.S. Pat. No. 5,846,797, where inventors disclosed processes for regeneration of plants from cotton callus via somatic embryogenesis; U.S. Pat. No. 5,846,797; U.S. Pat. No. 5,004,863; U.S. Pat. No. 5,159,135 and EP 344302 wherein inventors disclosed a method for transformation of cotton involving somatic embryogenesis based regeneration process and WO A1 9215675 wherein the inventor disclosed a method for bombardment mediated transformation of cotton embryonic axes wherefrom transgenic plants were developed.

The process described in the present invention is very simple to adopt commercially; fast, reproducible and convenient for applications in plant genetic engineering. Unlike state of the art technologies, this process does not lead to the formation of plants with morphological and cytogenetic abnormalities unlike in case of Stelly et al., 1989 and does not produce false positive transformants in transformation experiments unlike, in case of Sunilkumar and Rathore, 2001.

### OBJECTS OF THE INVENTION

It is an important object of the present invention to provide a method for developmentally synchronized somatic embryogenesis in cotton and sustained regeneration of a large number of plants from specified plant tissues.

Another object of the present invention is to provide an improved process for regeneration of cotton via somatic embryogenesis in a way that regenerated plants grow, mature and form fertile plants.

Yet another object of the present invention is to provide a simple and reproducible method for somatic embryogenesis with the least involvement of exogenously supplied plant growth regulators and any other extra media additive.

### SUMMARY OF THE INVENTION

The present invention provides for the first time an efficient method for plant regeneration in cotton via developmentally synchronized somatic embryogenesis. The process of this invention is simple, fast, reproducible and convenient for applications in plant genetic engineering. The most critical novel aspect is the achievement of synchronised somatic embryogenesis by a step of inositol starvation. This critical aspect is not mentioned or even suggested in any prior art known to the applicants.

The process of the present invention employs a growth regulator combination (2,4 dichlorophenoxy acetic acid and Benzyl adenine), different from those used in earlier reports, to induce calli from seedling explants. The process of the present invention achieves the production of callus mediated somatic embryogenesis that takes shorter time and gives a larger number of normal and fertile plants.

Thus, according to the present invention, there is provided a method for regenerating a large number of viable and fertile cotton plants via synchronized somatic embryogenesis from a hypocotyl segment or mesocotyl segment or a cotyledon piece, said method comprising—
(i) treating seeds of cotton plant with a sterilant to remove any unwanted contaminant.
(ii) culturing the treated seeds from step (i) for germination in a first medium consisting of:
   (a) salts of any conventional medium,
   (b) vitamins of any conventional medium,
   (c) inositol, and
   (d) carbon-source
   at a pH in the range of 5.2-6.0 and sterilizing the medium by autoclaving, incubating the cultures at the temperature 23-33° C. in light (in 30-60 μmol/m²/s intensity) or in darkness for a period of 6-12 days.
(iii) culturing explants from the seedlings obtained in step (ii).
(iv) culturing the explanst obtained from step (iii) for the purpose of callus induction in a second, solid medium consisting of
   (a) salts of any conventional medium,
   (b) vitamins of any conventional medium,
   (c) inositol,
   (d) carbon source,
   (e) Plant growth regulators in combination of 2, 4D and BA and
   (f) gelling agent
   at a pH in the range of 5.4 to 6.2 and sterilizing the medium by autoclaving, incubating the cultures in the range of 23-33° C. in light of at least 90 μmol/m²/s intensity with 16 h photoperiod;
(v) continuing the culturing for a period of 3-5 weeks till calli over the cut edges are formed.
(vi) transferring the calli generated from step 2 to a third, liquid medium at a packing density of 600-1000 mg callus/50 ml of medium, said medium comprising of:—
   (a) salts of any conventional medium,
   (b) vitamins of any conventional medium,
   (c) inositol and
   (d) carbon source
   at a pH in the range of 5.2-6.0 and sterilizing the medium by autoclaving, and incubating the cultures at a temperature between 23-33° C. in light intensity of 20-40 μmol/m²/s with a 16 h photoperiod for a period of 12-32 days, sufficient to form embryogenic clumps.
(vii) screening the cell suspension through metal sieves of different mesh sizes and selecting the cells/clumps collected over mesh size 40 and further subculturing the selected clumps to liquid basal medium as in step (vi).
(viii) subculturing embryogenic cells/clumps for a short period (8-12 days) to liquid basal medium of step (vi) but with no inositol, i.e. fourth medium.
(ix) further subculturing the embryogenic cells/clumps to liquid basal medium of step (vi) at a regular interval of 8-12 days.
(x) incubating the cultures in steps (viii) and (ix) at the same temperature, light, photoperiod condition as in step (vi).
(xi) shaking the cultures in step (vi), (viii) & (ix) at 110-130 rpm on a gyratory shaker.
(xii) transferring bipolar, mature somatic embryos on a fifth embryo germination medium comprising:—
   (a) salts of any conventional medium,
   (b) vitamins of any conventional medium,
   (c) inositol reduced to one fourth of normal concentration and
   (d) carbon-source,
   at a pH in the range of 5.2-6.0 and sterilizing the medium by autoclaving and incubating the cultures at a temperature between 23-33° C. in light intensity of at least 60 μmol/m²/s with a 16 h photoperiod till plantlets are developed.

In the present invention, the expression explants refer to cotyledon pieces or hypocotyl or mesocotyl segments.

In a preferred embodiment of the present invention, said first to fifth medium comprise salts of MS medium, vitamins of Gamborg B5 medium and a carbon source.

The first and fifth medium comprise salts of MS medium and vitamins of Gamborg B5 medium at half of its standard concentration, while second third and fourth medium comprise them in their standard concentration. The most preferred salts of MS medium and its standard concentration comprise the following as shown in Table 2:

TABLE 2
Salts of Murashige and Skoog (1962) medium:-

| Component | Conc. (mg/L) |
|---|---|
| NH₄NO₃ | 1650 |
| KNO₃ | 1900 |
| CaCl₂.2H₂O | 440 |
| MgSO₄.7H₂O | 370 |
| KH₂PO₄ | 170 |
| KI | 0.83 |
| H₃BO₃ | 6.2 |
| MnSO₄.4H₂O | 22.3 |
| ZnSO₄.7H₂O | 8.6 |
| Na₂MoO₄.2H₂O | 0.25 |
| CuSO₄.5H₂O | 0.025 |
| CoCl₂.6H₂O | 0.025 |
| Na₂.EDTA | 37.3 |
| FeSO₄.7H₂O | 27.8 |

The preferred vitamins of Gamborg B5 medium comprise the following as shown in Table 3:

TABLE 3

| Component | Conc. (mg/L) |
|---|---|
| Nicotinic Acid | 1.0 |
| Pyridoxine HCl | 1.0 |
| Thiamine HCl | 10 |

The preferred carbon source in the first medium is selected from a group consisting of sucrose and glucose and such carbon source employed is at a range of 1-3% wt./vol.

The preferred carbon source in the second, third and fourth medium is essentially glucose and such carbon source employed is at a range of 1.5-45. % wt./vol.

The preferred carbon course in the fifth medium is essentially sucrose and such carbon employed is at a range of 1-3% wt./vol. The preferred gelling agent in second medium is selected from a group consisting of agar (employed at a range of 0.6-0.8% wt./vol.) and phytagel (employed at a range of 0.15-0.29% wt./vol.). The preferred organics in first, second and third and fifth medium is essentially myo inositol, employed at 100 mg/L in first to third medium and at 25 mg/L in fifth medium.

The plant growth regulators employed in the second medium is selected from a group consisting of combinations of 2, 4D as auxin and BA as cytokinin.

In another preferred embodiment, the method of the present invention comprises:—
(i) sterilizing the seeds of cotton plant to remove the contaminants such as bacteria/fungus by conventional methods
(ii) culturing the sterilized seeds for germination in a medium shown in Table 4
(iii) excising the explants from the seedlings obtained in step (ii)
(iv) culturing the explant obtained in step (iii) in a medium as shown in Table 5 at a pH in the range of 5.4 to 6.2 and sterilizing the medium by autoclaving
(v) culturing the explants at the temperature of 23-33 degree C., light of at least 90 μmol/m²/s under 16 h photoperiod for a period of 3-5 weeks till sufficient calli form
(vi) transferring the calli to embryogenesis induction medium shown in Table 6 with the following composition at a pH in the range of 5.2-6.0 and sterilizing the medium by autoclaving
(vii) containing the culturing at the temperature of 23-33° C., light intensity in the range of 20-40 μmol/m²/s under 16 h photoperiod for a period of 2-5 weeks utill embryogenic clumps form
(viii) screening the embryogenic clump and transferring to inositol deprivation medium having the composition shown in Table 7 at a pH in the range of 5.2-6.0 and sterilizing the medium by autoclaving and culturing at the temperature of 20-40 μmol/m²/s under 16 h photoperiod for a short period of 8-12 days
(ix) transferring the osmotically shocked culture to liquid basal medium of step (vi) and continuing the culturing on this medium where somatic embryos synchronize
(x) transferring the mature bipolar somatic embryos to embryo germination medium as shown in Table 8 at a pH in the range of 5.2-6.0 and sterilizing the medium by autoclaving.
(xi) containing the culturing at the temperature of 23-33 degree c in light of at least 60 μmol/m²/s under 16 h photoperiod till plantlets are sufficiently developed to be taken out for acclimatization.

(xii) acclimatizing the regenerated plants in a potting mix comprising of garden soil:sand:vermiculite:pectmoss in 2:1:1:1 ratio

TABLE 4
Seed germination medium: -

| | |
|---|---|
| Component | Conc. (mg/L) |

a. Major salts

| | |
|---|---|
| NH₄NO₃ | 825 |
| KNO₃ | 950 |
| CaCl₂.2H₂O | 220 |
| MgSO₄.7H₂O | 185 |
| KH₂PO₄ | 85 |

b. Minor salts

| | |
|---|---|
| KI | 0.425 |
| H₃BO₃ | 3.6 |
| MnSO₄.4H₂O | 11.15 |
| ZnSO₄.7H₂O | 4.3 |
| Na₂MoO₄.2H₂O | 0.125 |
| CuSO₄.5H₂O | 0.0125 |
| CoCl₂.6H₂O | 0.0125 |
| Na₂.EDTA | 18.65 |
| FeSO₄.7H₂O | 13.9 |

c. Organics

| | |
|---|---|
| Myo-inositol | 100 |

d. Vitamins

| | |
|---|---|
| Nicotinic Acid | 0.5 |
| Pyridoxine HCl | 0.5 |
| Thiamine HCl | 5 |

e. Carbon source

Sucrose or Glucose 20 g/l

at a pH in the range of 5.2-6.0 and sterilizing the medium by autoclaving

TABLE 5
Callus induction medium

| | |
|---|---|
| Component | Conc. (mg/L) |

a. Major salts

| | |
|---|---|
| NH₄NO₃ | 1650 |
| KNO₃ | 1900 |
| CaCl₂.2H₂O | 440 |
| MgSO₄.7H₂O | 370 |
| KH₂PO₄ | 170 |

b. Minor salts

| | |
|---|---|
| KI | 0.83 |
| H₃BO₃ | 6.2 |
| MnSO₄.4H₂O | 22.3 |
| ZnSO₄.7H₂O | 8.6 |
| Na₂MoO₄.2H₂O | 0.25 |
| CuSO₄.5H₂O | 0.025 |
| CoCl₂.6H₂O | 0.025 |
| Na₂.EDTA | 37.3 |
| FeSO₄.7H₂O | 27.8 |

c. Organics

| | |
|---|---|
| Myo-inositol | 100 |

d. Vitamins

| | |
|---|---|
| Nicotinic Acid | 1.0 |
| Pyridoxine HCl | 1.0 |
| Thiamine HCl | 10 |

e. Carbon source

Glucose 30 g/l

f. Gelling agent

Agar 8g/l or Phytagel 2.2 g/l

g. Plant Growth Regulators

Auxins 0.44 to 4.4 μM
Cytokinins 0.22 μM to 2.2 μM

TABLE 6
Embryogenesis induction medium

| | |
|---|---|
| Component | Conc. (mg/L) |

a. Major salts

| | |
|---|---|
| NH₄NO₃ | 1650 |
| KNO₃ | 1900 |
| CaCl₂.2H₂O | 440 |
| MgSO₄.7H₂O | 370 |
| KH₂PO₄ | 170 |

b. Minor salts

| | |
|---|---|
| KI | 0.83 |
| H₃BO₃ | 6.2 |
| MnSO₄.4H₂O | 22.3 |
| ZnSO₄.7H₂O | 8.6 |
| Na₂MoO₄.2H₂O | 0.25 |
| CuSO₄.5H₂O | 0.025 |
| CoCl₂.6H₂O | 0.025 |
| Na₂.EDTA | 37.3 |
| FeSO₄.7H₂O | 27.8 |

c. Organics

| | |
|---|---|
| Myo-inositol | 100 |

d. Vitamins

| | |
|---|---|
| Nicotinic Acid | 1.0 |
| Pyridoxine HCl | 1.0 |
| Thiamine HCl | 10 |

e. Carbon source

Glucose 30 g/l

TABLE 7
Inositol deprivation medium

| | |
|---|---|
| Component | Conc. (mg/L) |

a. Major salts

| | |
|---|---|
| NH₄NO₃ | 1650 |
| KNO₃ | 1900 |
| CaCl₂.2H₂O | 440 |
| MgSO₄.7H₂O | 370 |
| KH₂PO₄ | 170 |

b. Minor salts

| | |
|---|---|
| KI | 0.83 |
| H₃BO₃ | 6.2 |
| MnSO₄.4H₂O | 22.3 |
| ZnSO₄.7H₂O | 8.6 |
| Na₂MoO₄.2H₂O | 0.25 |
| CuSO₄.5H₂O | 0.025 |
| CoCl₂.6H₂O | 0.025 |
| Na₂.EDTA | 37.3 |
| FeSO₄.7H₂O | 27.8 |

c. Vitamins

| | |
|---|---|
| Nicotinic Acid | 1.0 |
| Pyridoxine HCl | 1.0 |
| Thiamine HCl | 10 |

d. Carbon source

Glucose 30 g/l

TABLE 8
Embryo germination medium

| | |
|---|---|
| Component | Conc. (mg/L) |

a. Major salts

| | |
|---|---|
| NH₄NO₃ | 825 |
| KNO₃ | 950 |
| CaCl₂.2H₂O | 220 |
| MgSO₄.7H₂O | 185 |
| KH₂PO₄ | 85 |

b. Minor salts

| | |
|---|---|
| KI | 0.425 |
| H₃BO₃ | 3.6 |
| MnSO₄.4H₂O | 11.15 |
| ZnSO₄.7H₂O | 4.3 |
| Na₂MoO₄.2H₂O | 0.125 |
| CuSO₄.5H₂O | 0.0125 |
| CoCl₂.6H₂O | 0.0125 |
| Na₂.EDTA | 18.65 |
| FeSO₄.7H₂O | 13.9 |

c. Organics

| | |
|---|---|
| Myo-inositol | 25 |

d. Vitamins

| | |
|---|---|
| Nicotinic Acid | 0.5 |
| Pyridoxine HCl | 0.5 |
| Thiamine HCl | 5 |

e. Carbon source

Sucrose 20 g/l

According to an embodiment of the invention, the basal embryogenic mass can be subjected to further rounds of inositol starvation and subsequent synchronized embryogenesis and regeneration of plans.

### DETAILED DECSRIPTION

In the method of the present invention, seeds are surface sterilized before use in in vitro culture to make them free of bacterial/fungal contaminant. Surface sterilization involves treating the seeds with a solution containing any one of sterilizing agent such as sodium hypochlorite, calcium hypochlorite, mercuric chloride alcohol cetrimide etc. The surface sterilization of seeds can be performed by treating the seeds with 0.05-0.5% w/v solution of mercuric chloride in water for 3-11 minutes with continuous swirling and then washing thoroughly with sterile distilled water (4-8 times) followed by dipping the seeds in rectified spirit (50-100% v/v) for 10-20 sec and then scorching it in the flame of a spirit burner for 5-10 sec.

Surface sterilized seeds can be placed for germination on filter paper boats moistened with seed germination medium containing Murashige and Skoog salts at half of its concentration, Gamborg B5 medium at half of its concentration, 100 mg/L inositol and any carbon source like glucose or sucrose 1 to 3% wt./vol., adjusting the pH of the medium to 5.2-6.0 and sterilized as a result of autoclaving at 121 degree c, 16 psi for 16 min.

For germination, seeds may be incubated at temperature 23-33° C. in light (in 30-60 μmol/m²/s intensity) or in darkness till seed germinates and form a mature seedling.

The explants (cotyledon pieces, hypocotyl segments or mesocotyl segments) can preferably be obtained from 6-12 d old seedling after germination by cutting with a sharp sterile scalpel and blade in an aseptic environment i.e. laminar flow, known in the art.

The excised explants can be placed in the medium containing salts of Murashige and Skoog at concentrations as given in table 2, vitamins of Gamborg B5 at concentrations as given in table 3, 100 mg/L inositol, a carbon source preferably glucose 1.5-4.5% wt./vol., a gelling agent preferably agar 0.6 to 0.8% wt./vol. or phytagel 0.15-0.29% wt./vol. and plant growth regulators 2, 4D 0.44 to 4.44 μM and BA 0.22 to 2.22 μM. The medium pH is adjusted to 5.4-6.2 prior to autoclaving at 121° C., 16 psi for 16 min. Composition of the medium provided for callus induction is presented in Table 5 Cultures were incubated at temperature in the range of 23-33° C. in white fluorescent light of at least 90 μmol/m²/s intensity under 16 h photoperiod for a period of 3-5 weeks. By this time, sufficient calli form over the cut edges of the explants. The calli can be yellowish to brownish in appearance and friable in texture.

The calli, developed over the cut edges of the explant, can be transferred to a liquid basal medium, the composition of which is given in table 6 at a packing density of 600 to 1000 mg of callus per 50 ml of media in a 250 ml Ehrleneyer flask. The medium does not contain any growth regulator or gelling agent and its pH is adjusted to 5.2-6.0 prior to autoclaving at 121° C., 16 psi for 16 min. The cells of the calli can be agitated in this medium at 110-130 strokes per minute on a gyratory shaker set at 23-33° C. temperature, 20-40 μmol/m²/s light intensity under 16 hrs. photoperiod. The cells can be cultured in this medium and incubation conditions for a period of 12-32 days, till embryogenic clumps form in the cell suspension culture.

The cell suspension developed in agitating liquid basal medium can be screened to select embryogenic clumps/group of cells developed in the culture. It can be passed through a combination of metal sieves of mesh sizes 10, 40 and 100. The mesh size 10 collects bigger tissue clumps and mesh size 100 collects fine suspension of cells. The fraction of cells containing smaller clumps, which may turn embryogenic can be collected over sieve with mesh size 40. The selected fraction of cells can be transferred to the fresh liquid basal medium and regularly subcultured at an interval of 8-12 days or can be transferred to the inositol deprivation medium which is liquid basal medium minus inositol, the composition given in Table 7, for a period of 8-12 days followed by replacing inositol in fresh liquid basal medium and then regularly subcultured at an interval of 8-12 days. In both cases somatic embryos develop but with different developmental fate. While in the former, embryos of all developmental stages can be obtained in each subculturing cycle of 8-12 days with similar frequency, in the latter, embryos development gets synchronized and almost all embryos remain at same developmental stage. The strategy in the present method is to select second approach to culture the embryogenic mass.

Furthermore, after the mature bipolar torpedo stage embryos taken out of suspension for germination, the basal embryogenic mass can be subjected for further cycles of inositol depletion, developmental synchronization and subsequent harvest of mature embryos for germination.

The embryogenic clumps, and synchronized embryos can be cultured in agitating medium on a gyratory shaker at 110-130 strokes per minute and at 23-33° C. temperature, 20-40 μmol/m²/s light under 16 hrs. photoperiod.

The mature bipolar somatic embryos can be taken out of liquid medium and transferred to a solid support, preferably vermiculite saturated with embryo germination medium composition of which is given in table 8. The medium pH is adjusted to 5.2-6.0 prior to autoclaving at 121° C., 16 psi for 16 minutes. The cultures can be incubated at 23-33° C. temperature in light intensity of at least 60 μmol/m²/s and a photoperiod of 16 hr. The fresh liquid medium can be added to the germinating embryos on weekly basis. The embryos were grown in embryo germination medium for a period of time sufficient to form 4-5 leaf stage plantlet with well-developed roots. At this stage, plantlets can be taken out and may be transferred to potting mix which is a sterile mixture of garden soil:sand:vermiculite:peatmoss in 2:1:1:1 ratio. Good humid conditions can be given to the newly developed plantlets by covering the plants with transparent polythene bags the inner surface of which is drizzled with water. After culturing the plantlets under these conditions and at temperature of 23-33 degree C. and fluorescent light of at least 90 μmol/m²/s under a 16 h photoperiod for a period of time sufficient for acclimatization, the plants, if desired, can be transferred to the field.

The present invention will now be described in greater detail with reference to the following non-limiting examples.

### EXAMPLE 1

Media Dependent Response of Explants to Induction of Somatic Embryogenesis

Seeds of cotton (G. hirsutum L. Coker 312) plants were treated with 0.1% w/v mercuric chloride for 7 minutes, washed with sterile distilled water 6 times, followed by dipping the seeds in rectified spirit for 10 seconds and flamed. The sterile seeds were placed for germination on filter paper boats moistened with seed germination medium containing Murashige and Skoog salts at half of its concentration, Gamborg B5 vitamins at half of its concentration, 100 mg/l inositol and 2% w/v sucrose (pH of the medium was adjusted to 5.6 before autoclaving). For germination, seeds were incubated at 28±2° C. temperature in white fluorescent light (30 μmol/m²/s) under 16 h photoperiod. The culture was continued till seeds germinate to give radicle and plumule with well-expanded cotyledons.

Nine-day-old seedlings were used to provide the hypocotyl segments and cotyledon pieces as explants. Explants were excised with the help of a sharp, sterile scalpel. The explants were placed on the callus induction medium, CIM1 containing Murashige and Skoog salts, Gamborg B5 vitamins, 100 mg/l inositol, 3% w/v glucose, 750 mg/l MgCl₂ and 0.22% w/v phytagel, supplemented with 2.2 μM 2,4-D and 0.88 μM BA (pH of the medium was adjusted to 5.8 before autoclaving).

The explants were incubated on this medium at 28±2° C. temperature in 90 μmol/m²/s light intensity under 16 h photoperiod for 3-4 weeks. The calli developed over the cut edges of the explants were excised and inoculated in a liquid basal medium containing Murashige and Skoog salts, Gamborg B5 vitamins, 100 mg/l inositol, 3% w/v glucose, pH 5.6 at a packing density of 800 mg callus per 50 ml medium in 250 ml Ehrlenmeyer flasks. The medium was sterilized by autoclaving at 121° C., 16 psi for 16 minutes. The cultures were agitated on a gyratory shaker at 120 rpm and at 28±2° C. temperature, 30 μmol/m²/s light intensity under 16 h photoperiod.

Culturing of the explants for callus induction was also done on media CIM2, CIM3 and CIM4 containing Murashige and Skoog salts, Gamborg B5 vitamins, 100 mg/l inositol, 3% w/v glucose, 750 mg/l MgCl₂ and 0.22% w/v phytagel supplied with different growth regulator combinations, like 0.45 μM 2,4-D plus 2.32 μM Kin. (in CIM2); 10.7 μM NAA plus 4.64 μM Kin. (in CIM3); 2.68 μM NAA plus 2.4 μM 2iP (in CIM4). Cultures were incubated in the same temperature and light conditions. The calli were transferred to the liquid basal medium and cultured in a similar way.

After 20-22 days growth of culture in liquid medium, the suspension generated thereof was screened through metal sieves of different pore sizes (mesh 10, 40 and 100, from SIGMA chemical company, St. Louis). The bigger clumps of cells collected over mesh size 10 and fine suspension collected over mesh size 100 were discarded. The smaller clumps of cells collected on mesh size 40 were subcultured to fresh liquid basal medium and scored for the presence of somatic embryos. Somatic embryogenesis (SE) induction frequency was calculated on the basis of presence of embryogenic clumps and the number of somatic embryos obtained per explant was scored accordingly in each media combination. The results obtained are summarized in Table 9.

TABLE 9
Embryogenesis response of different explant types on the selected
media compositions

| | SE induction | |
|---|---|---|
| | (% frequency* ± | Mature embryos per |
| Media combinations | S.E.) | explant (number + S.E.) |
| Cotyledon explants | | |
| CIM1 | 71.66 ± 10.40 | 20.33 ± 2.02 |
| CIM2 | 56.66 ± 5.77 | 16.05 ± 1.73 |
| CIM3 | 33.30 ± 14.43 | 13.00 ± 2.64 |
| CIM4 | 58.33 ± 7.55 | 14.72 ± 1.10 |
| Hypocotyl explants | | |
| CIM1 | 78.30 ± 7.95 | 20.00 ± 2.16 |
| CIM2 | 62.90 ± 10.56 | 15.24 ± 1.96 |
| CIM3 | 34.15 ± 12.28 | 12.33 ± 1.82 |
| CIM4 | 56.65 ± 8.13 | 17.40 ± 1.49 |

*% explants that show induction of embryogenic callus.

Critical difference for somatic embryogeness (SE) induction and mature embryo formation were 17.69 and 3.43 for cotyledon (n=5, repeated twice) and 13.14 and 2.16 for hypocotyl (n=6, repeated thrice) explants respectively.

It is apparent from the results that variations in combinations and concentrations of growth regulators give different response with respect to somatic embryogenesis induction frequency and number of mature somatic embryos obtained per explant. Thus, this finding can be utilized for developing the most optimal conditions for regeneration of cotton plant.

### EXAMPLE 2

The procedure of Example 1 was repeated except that the seeds were germinated and grown for 9 days until radicle and plumule develop with well expanded cotyledons under dark. Essentially the same results were obtained.

### EXAMPLE 3

The procedure of Example 1 was repeated except that the seed germination medium contained 2% w/v glucose as the carbon source. The same results were obtained.

### EXAMPLE 4

The procedure of Example 2 was repeated except that the seed germination medium contained 2% glucose in place of sucrose. The similar results were obtained.

### EXAMPLE 5

The procedures of Examples 1 and 2 were repeated with cotton variety Coker 310. Similar results were obtained.

### EXAMPLE 6

The procedure of Example 1 was repeated to the extent of obtaining embryogenic clumps in suspension derived from callus generated on a combination of 2,4-D and BA. The embryogenic clumps were subcultured in the liquid basal medium (BM) devoid of inositol. The embryogenic clumps subcultured to liquid basal medium containing inositol was used as control. The clumps were inoculated at a packing density of approximately 800 mg of cells per 50 ml medium and incubated on a gyratory shaker at the same light, temperature and photoperiod conditions as in Example 1. After 1 to 2 subculturing of 10 days in medium devoid of inositol, the cultures were returned to inositol containing basal liquid medium. The frequency and the number of embryos in different developmental stages per unit mass were scored in both conditions. The data obtained is presented in Table 10

TABLE 10
Synchronization of somatic embryogenesis by inositol starvation

| | No. of embryos in | No. of embryos in heart | No. of embryos in |
|---|---|---|---|
| Treatments | globular stage (%) | stage (%) | torpedo stage (%) |
| No starvation cycle: | | | |
| (i) 1 subculture to Basal | 24.00 ± 8.00 (46.18%) | 18.64 ± 6.08 (35.87%) | 9.32 ± 2.28 (17.93%) |
| Medium (BM) | | | |
| (ii) 2 subcultures to BM | 37.32 ± 6.08 (56.03%) | 18.64 ± 4.6 (27.98%) | 10.64 ± 6.08 (15.97%) |
| (iii) 3 subcultures to BM | 20.00 ± 6.92 (38.49%) | 17.32 ± 4.6 (33.3%) | 14.64 ± 6.08 (28.17%) |
| 1 cycle of starvation, | | | |
| followed by: | | | |
| (i) 1 subculture to BM | Numerous | — | — |
| (ii) 2 subcultures to BM | — | 132.00 ± 22.24 (91.6%) | 12.00 ± 4.00 (8.3%) |
| (iii) 3 subcultures to BM | — | 25.32 ± 8.32 (18.09%) | 114.64 ± 12.2 (81.9%) |
| 2 cycles of starvation, | | | |
| followed by | | | |
| (i) 1 subculture to BM | Numerous | — | — |
| (ii) 2 subcultures to BM | — | 34.64 ± 8.32 (74.27%) | 12.00 ± 4.00 (25.72%) |
| (iii) 3 subcultures to BM | — | 36.00 ± 4.00 (49.09%) | 37.32 ± 12.84 (50.9%) |

It is clear from the results that without the inositol depletion, non-synchronised embryogenesis was obtained. Therefore, continuous subculturing of the embryogenic mass to the basal medium containing inositol gave embryos in all developmental stages at nearly similar frequency. However, when embryogenic mass were subcultured to inositol free basal medium for a single cycle of 10 days and then returned to basal medium containing inositol, it produced a synchronized development of somatic embryos (100% embryos in globular stage after one subculture to basal medium; 91.66% embryos in heart stage after 2 subcultures to basal medium and 81.99% embryos in torpedo stage after 3 subcultures). The total number of mature embryos produced per explant was increased to a 4-5 fold higher value. The embryogenic clumps, when subcultured to an inositol-free medium for 2 cycles showed a reduction in homogeneity in the developmental stages of embryos. Hence, single cycle of inositol starvation was required for inducing a high level of synchrony in embryogenesis.

### EXAMPLE 7

Seeds of cotton (G. hirsutum L. Coker 312) plants were sterilized and germinated as in Example 1. The explants were dipped in Agrobacterium suspension for 10-15 minutes, blot dried and inoculated on a co-cultivation medium comprising Murashige and Skoog salts, Gamborg B5 vitamins, 100 mg/l inositol, 3% w/v glucose, 750 mg/l MgCl₂ and 0.22% w/v phytagel, supplemented with 2.2 μM 2,4-D and 0.88 μM BA (pH of the medium was adjusted to 5.8 before autoclaving). The cultures were incubated at 28±2° C. temperature in 90 μmol/m²/s light intensity under 16 h photoperiod for 3 days. After 3 days explants were washed with sterile water, blot dried again and inoculated on the medium having a composition similar to co-cultivation medium except that it was further supplemented with 250 mg/l augmentin and 50 mg/l kanamycin. The explants were further cultured as according to Example 1 and Example 6. Similar results were obtained with respect to somatic embryogenesis and regeneration of transformed plants.

The Agrobacterium strain utilized in this experiment was a common laboratory strain LBA 4404 harboring derivative of a binary vector pIG which had nptII as selection marker and gusA with an intron as a reporter gene.

It is clear from the regeneration obtained with transformed explants that culturing the putatively transformed cells in liquid medium in presence of selection agent (kanamycin in this case) is less prone to produce false positive selectants as all the embryos recovered were GUS +ve and showed homogeneous blue colour after histochemical assay. Furthermore, somatic embryogenesis induction frequency and number of mature somatic embryos per explant were obtained with the transformed explants, and the results were similar, with as well as without treatment with Agrobacterium tumefaciens.

### EXAMPLE 8

The procedure of Example 7 was repeated except that explants were co-cultivated under dark. Essentially similar results were obtained.

1. A method for regeneration of cotton via somatic embryogenesis with substantially synchronized development of embryos after short duration inositol starvation, said process comprising the steps of:
(i) cutting from the germinated cotton seedling the explant, selected from a group consisting of cotyledon, hypocotyl, mesocotyl and mixtures thereof;
(ii) culturing the explant for the purpose of callus induction in a first solid medium, in a culture medium containing glucose as the carbon source supplemented with Gamborg B5 vitamins, 2,4-D and BA and inositol, at a temperature between 23 to 33° C. in light intensity of at least 90 μmol/m²/s under a 16 hour photopeiod for a period of 3-5 weeks, to enable dedifferentiated callus to form from the explant;
(iii) transferring the callus from the first solid callus induction medium to a liquid medium comprising a basal medium containing glucose as the carbon source and supplemented with Gamborg B5 vitamins and culturing the suspension generated thereof at a temperature from 23 to 33° C. in a reduced light intensity of 20-40 μmol/m²/s, under a 16 hour photoperiod for a period of time sufficient to form embryogenic clumps;
(iv) screening the cell suspension through metal sieves of different pore sizes to select embryogenic cells and/or clumps and subculturing the embryogenic callus containing somatic embryos to said basal medium;
(v) subjecting the embryogenic mass/clumps to inositol deprivation, consequent upon subculturing it to said basal medium devoid of inositol for a sufficient period of time and then returning the culture to inositol containing medium to enable the somatic embryos to synchronize developmentally;
(vi) transferring bipolar somatic embryos to an embryo germination medium on a support and growing the embryos in embryo germination medium up to the plantlet stage developed sufficiently for transfer to soil;
(vii) further transferring the plantlets to a potting mix for acclimatization and then to field.

2. A method as claimed in claim 1, wherein the explants are derived from cotton or any other plant seedlings.

3. The method as recited in claim 1, wherein the explant is derived from cotton cv Coker 312 and the seedlings are developed by:
(i) sterilizing cotton seed in a sterilization solution of 0.1% HgCl₂ for 5-10 min., preferably 7 min.,
(ii) rinsing the seed in sterile water 4-6 times,
(iii) scorching the seed in flame of a spirit burner for 5-10 seconds,
(iv) inoculating the seed on seed germination medium,
(v) growing the seed in the seed germination medium in light or dark at a temperature of 23 degree to 33 degree C. for a period of 6-12 days, preferably 9-10 days,
(vi) excising the explant from the seedling.

4. A method as claimed in claim 3, wherein seed germination medium is a liquid medium comprising salts of Murashige and Skoog and Gamborg B5 vitamins at half of its concentration.

5. A method as claimed in claim 3, wherein carbon source in seed germination medium is selected from a group consisting of sucrose and glucose at a range of 1 to 3% wt./vol.

6. A method as claimed in claim 1, wherein said first solid callus induction medium comprises following components of Murashige and Skoog medium:

| | |
|---|---|
| Component | Conc. (mg/L) |

a. Salts of Murashige and Skoog (1962) medium: -

| | |
|---|---|
| NH₄NO₃ | 1650 |
| KNO₃ | 1900 |
| CaCl₂.2H₂O | 440 |
| MgSO₄.7H₂O | 370 |
| KH₂PO₄ | 170 |
| KI | 183 |
| H₃BO₃ | 6.2 |
| MnSO₄H₂O | 22.3 |
| ZnSO₄.7H₂O | 8.6 |
| Na₂MoO₄.2H₂O | 0.25 |
| CuSO₄.5H₂O | 0.025 |
| CoCl₂.6H₂O | 0.025 |
| Na₂.EDTA | 37.3 |
| FeSO₄.7H₂O | 27.8 |

b. Organics

| | |
|---|---|
| Myo-inositol | 100 |

7. A method as claimed in claim 1, wherein Gamborg B5 vitamins, wherever included comprise:

| Component | Conc. (mg/L) |
|---|---|
| Nicotinic Acid | 1.0 |
| Pyridoxine HCl | 1.0 |
| Thiamine HCl | 10 |

8. A method as claimed in claim 1, wherein 2,4-D as exogenously supplied auxin in first solid callus induction medium is selected from a range of 0.44 to 4.4 μM, preferably 1.76 to 2.64 μM.

9. A method as claimed in claim 1, wherein BA as exogenously supplied cytokinin in first solid callus induction medium is selected from a range of 0.22 μM to 2.2 μM, preferably 0.66 μM to 1.00 μm.

10. A method as claimed in claim 1, wherein gelling agent in said first solid callus induction medium is selected from a group consisting of agar in the range of 0.6-0.8% wt./vol., preferably 0.7% and phytagel in the range of 0.15-0.29% wt./vol., preferably 0.22% wt./vol.

11. A method as claimed in claim 1, wherein said first solid callus induction medium contains glucose as the primary carbon source.

12. A method as claimed in claim 1, wherein said explants are cultured on said callus induction medium at a temperature between 23 to 33° C., preferably between 27 to 29° C. in light intensity of at least 90 μmol/m²/s under a 16 h photoperiod for period of not more than of 3-5 weeks, to enable dedifferentiated callus to form from any of the said explant.

13. A method as claimed in claim 1, essentially including the step of transferring callus from the said first solid callus induction medium to a liquid medium in Ehrlenmeyer flasks at a packing density of 600 to 1000 mg of callus/50 ml of media preferably, 800 mg/50 ml and shaking the culture in this and all subsequent steps until somatic embryos are taken out for germination on a gyratory shaker at 110-130 rpm.

14. A method as claimed in claim 1, wherein said embryogenesis induction medium is a basal liquid medium comprising M&S salts, Gamborg B5 vitamins, inositol and glucose as the carbon source.

15. A method as claimed in claim 1, wherein plant cell suspension embryogenic mass and somatic embryos generated thereof in liquid medium are incubated at a temperature from 23 to 33° C., preferably 27-29° C. in light intensity of 20-40-μmol/m²/s, typically 27-33 μmol/m²/s under a 16 h photoperiod.

16. A method as claimed in claim 1, wherein said embryogenic mass/clumps are subjected to inositol deprivation for a period of 8 to 12 days, preferably, 10 days in inositol deprivation medium comprising MS basal salts, Gamborg B5 vitamins, glucose as carbon source but no inositol, leading to developmental synchronization of somatic embryos.

17. A method as claimed in claim 1, wherein said first solid callus induction medium has a pH in the range of 5.4-6.2 and the entire liquid media in said process has a pH in the range of 5.2-5.8, being sterile as a result of autoclaving at 121° C., 16 psi for 16 minutes.

18. A method as claimed in claim 1, wherein potting mix comprises of garden soil:sand:Peat moss:vermiculite typically in 2:1:1:1 ratio.

19. A method as claimed in claim 1, wherein developmental synchrony of somatic embryogenesis is utilized for multiplication of elite cotton cultivar or development of transgenic cotton cultivar.

20. A method as claimed in claim 1, wherein the inositol depletion is applied to plant species other than cotton for enhancing embryogenesis in tissue culture.

21. A method as claimed in claim 1, wherein said culture medium and basal medium comprise of Murashige and Skoog medium.

22. A method as claimed in claim 1, wherein said period of time sufficient to from embryonic clumps comprises 12-32 days.

23. A method as claimed in claim 1, wherein said subculturing the embryogenic callus containing somatic embryos to said basal medium is carried out at intervals of 812 days.

24. A method as claimed in claim 1, wherein said embryogenic mass/clumps are subjected to inositol deprivation for a period of 8 to 12 days, preferably, 10 days.

25. A method as claimed in claim 1, wherein said support for said embryo germination medium comprises vermiculite.
